(19) United States
(12) Patent Application Publication
(10) Pub. No.: US 2008/0125654 A1
(43) Pub. Date: May 29, 2008

(54) ULTRASOUND SYSTEM

(75) Inventors: Dong Gyu HYUN, Seoul (KR); Mok Kun Jeong, Seoul (KR)

Correspondence Address: OBLON, SPIVAK, MCCLELLAND MAIER & NEUSTADT, P.C., 1940 DUKE STREET, ALEXANDRIA, VA, 22314, US

(73) Assignee: Medison Co., Ltd., Hongchun-gun (KR)

(21) Appl. No.: 11/930,960

(22) Filed: Oct. 31, 2007

(30) Foreign Application Priority Data
Nov. 27, 2006 (KR) 10-2006-0117466

(51) Int. Cl. A61B 8/00

(52) U.S. Cl. 600/443

(57) ABSTRACT

The present invention generally relates to an ultrasound system for diagnosing breast tumors without subjecting patients to pain. The ultrasound system of the present invention, comprises: a probe for transmitting ultrasound signals to a target and receiving the ultrasound signals reflected from the target, the probe being further configured to apply stress from a user to the target; a stress sensing unit disposed at the opposite side of the probe to support the target and sense the magnitude of the stress applied from the probe, the stress sensing unit being further configured to form stress magnitude information; a processor for forming elastic images based on the received signals and the stress magnitude information; and a display unit for displaying the elastic images processed by the processor.

The present application claims priority from Korean Patent Application No. 10-2006-0117466 filed on Nov. 27, 2006, the entire subject matter of which is incorporated herein by reference.

### BACKGROUND

1. Field

The present invention generally relates to an ultrasound system, and more particularly to an ultrasound system adapted to form elastic images for diagnosis of breast tumor.

2. Background

Breast cancer or adenocarcinoma of breast is a significant medical concern, especially for women. The breast cancer not only increases the mortality rate of women, but also leads to severe psychological/emotional trauma as well as economic loss. Thus, the prevention of breast tumors and regular periodic medical examinations to find such tumors at an early stage are very important. As such, there has been introduced a breast cancer diagnostic apparatus geared to performing early diagnosis of breast tumor. A conventional breast cancer diagnostic apparatus is explained in view of FIG. 1.

As shown in FIG. 1, a conventional breast cancer diagnostic apparatus 10 comprises: a body 11; a main post 12 installed along a longitudinal direction of the body 11 and engaged thereto so that it may rotate 360 degrees with a rotational axis 13 as a center; an x-ray set 14 with an x-ray tube head 15 fixed to an upper front side of the main post 12 such that it may ascend and descend; a compressing member 17 provided underneath the x-ray set 14 and in front of a sub post 16 configured to move up and down along a direction of the main post 12 so that it may slide up and down; and an image receptor 18 provided underneath the compressing member 17 and fixed to a lower side of the sub post 16 so that it may be adhered to the compressing member 17.

To examine whether a patient has breast cancer, the patient's breast is first supported by the image receptor 18. Then, the compressing member 17, which is movably installed to the sub post 16, is descended so that the patient's breast is pressed with a load of approximately 20 kg. Thereafter, the x-ray set 14 is descended and the portion of the breast necessary for diagnosis is imaged by the x-ray tube head 15.

However, since the conventional breast cancer diagnostic apparatus is adapted to press the patient's breast with a 20 kg load, it is only inevitable that the patient feels severe pain. Further, the patient's posture deteriorates due to such pain, thereby becoming extremely difficult to obtain accurate images.

In order to address and resolve the above-mentioned problems, the present invention provides an ultrasound system adapted to provide elastic images for diagnosing breast tumors based on the magnitude of stress applied through a probe.

### BRIEF DESCRIPTION OF THE DRAWINGS

Arrangements and embodiments may be described in detail with reference to the following drawings in which like reference numerals refer to like elements and wherein:

FIG. 1 is a perspective view illustrating a conventional breast cancer diagnostic apparatus;

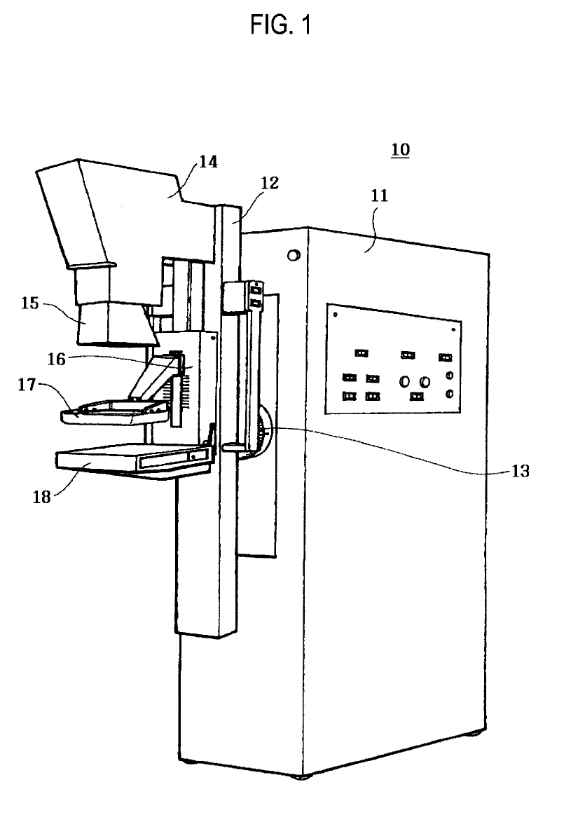

FIG. 2 is a perspective view illustrating an ultrasound system constructed in accordance with the present invention; and

FIG. 3 illustrates the use of the ultrasound system shown in FIG. 2.

### DETAILED DESCRIPTION

A detailed description may be provided with reference to the accompanying drawings. One of ordinary skill in the art may realize that the following description is illustrative only and is not in any way limiting. Other embodiments of the present invention may readily suggest themselves to such skilled persons having the benefit of this disclosure.

An ultrasound system constructed in accordance with a preferred embodiment of the present invention may include a probe, a stress sensing unit, a processor and a display unit. The probe is configured to transmit ultrasound signals to a target and receive the ultrasound signals reflected from the target. The probe may be further adapted to apply stress from a user to the target. The stress sensing unit may be arranged at the opposite side of the probe so as to support the target. Also, the stress sensing unit is configured to sense or detect the magnitude of the stress applied from the probe so that it may form stress magnitude information. Moreover, the processor is adapted to form elastic images based on the received signals and the stress magnitude information. The display unit may then display the elastic images.

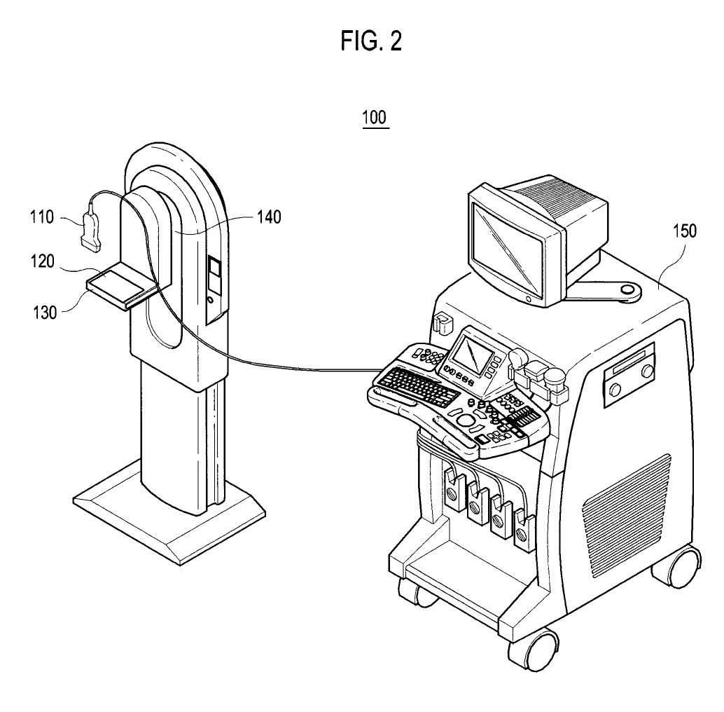

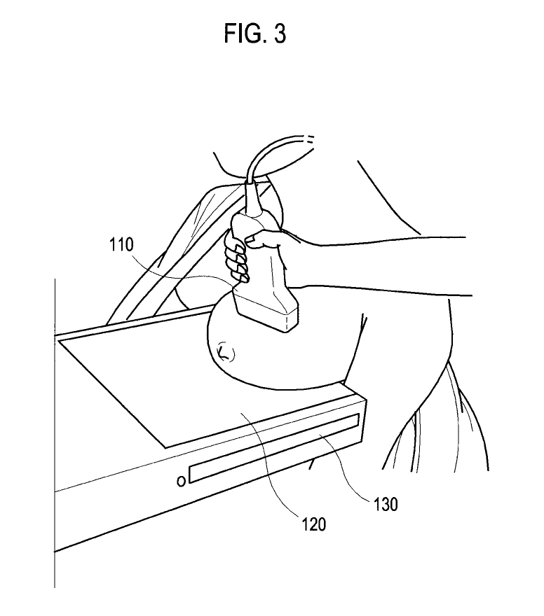

Hereinafter, the embodiments of the present invention are described in view of FIGS. 2 and 3.

As shown in FIG. 2, an ultrasound system 100 constructed in accordance with the present invention may include a probe 110, a stress sensing unit 120, a supporting unit 130, a moving unit 140 and an ultrasound diagnosis unit 150.

The probe 110 is designed to transmit ultrasound signals to a target and receive the ultrasound signals reflected from the target. The probe 110 may further apply stress from a user to the target. Alternatively, the probe 110 may further include a position sensor (not shown) for measuring the distance to the stress sensing unit 120.

The stress sensing unit 120 may be arranged at the opposite side of the probe so as to support the target. The stress sensing unit 120 may sense or detect the stress applied to the target through the probe 110 in order to form magnitude information of the stress. As shown in FIG. 3, the stress sensing unit 120 may preferably be in the form of a plate so that the stress applied from the probe 110 to the target (especially to the patient's breast) may be uniformly sensed or detected. The stress sensing unit 120 may be further adapted to transmit the formed stress magnitude information to the body 150 via wired or wireless means. Alternatively, the stress sensing unit 120 may further include an acoustic sensor (not shown) for sensing the ultrasound signals transmitted from the probe 110 and measuring the time duration taken for the ultrasound signals to reach the stress sensing unit 120.

The supporting unit 130 may be configured to support the stress sensing unit 120 so that the stress sensing unit 120 may sense the stress applied from the probe 110.

Under the control of the ultrasound diagnosis unit 150, the moving unit 140 may be configured to move the supporting unit 130 upward and downward so that the patient may conveniently place the target (i.e., breast) from a sitting position. In such a case, the height of the patient may be considered. Alternatively, the moving unit 140 may be moved upward and downward.

The ultrasound diagnosis unit 150 may be configured to control the moving unit 140 so that the supporting unit 130 may move upward and downward. The ultrasound diagnosis unit 150 may be further configured to form elastic images based on the signals received from the probe 110 and the stress magnitude information from the stress sensing unit 120. Although not shown in the provided figures, the ultrasound diagnosis unit 150 may include a beam former for transmit-focusing the ultrasound signals transmitted through the probe 110. The beam former may also be adapted for receive-focusing the ultrasound signals received through the probe 110. The ultrasound diagnosis unit 150 may further include a processor for forming elastic images based on the received signals and the stress magnitude information. The ultrasound diagnosis unit 150 may further include a display unit for displaying the elastic images processed by the processor. In such a case, the processor may compare the received signals in cases where the stress is applied and not applied. The processor may then calculate the strain factors of the target in both of the cases. The processor may be further adapted to calculate the elasticity modulus based on the calculated strain factor and the stress magnitude information. The processor may then form the elastic images by using the calculated elasticity modulus. In addition, the ultrasound diagnosis unit 150 may be configured to provide the distance information measured by the position sensor of the probe 110 and the arrival time information of the ultrasound signals measured by the acoustic sensor of the stress sensing unit 120 as additional information.

As described above, the ultrasound system of the present invention may provide elastic images for diagnosing breast tumors without applying any large load (approximately 20 kg) to the target (i.e., patients' breast). This allows the breast tumors to be diagnosed without subjecting the patients to any pain.

In accordance with one embodiment of the present invention, there is provided an ultrasound system, comprising: a probe for transmitting ultrasound signals to a target, receiving the ultrasound signals reflected from the target and applying stress to the target; a stress sensing unit disposed at the opposite side of the probe to support the target, sense the magnitude of the stress applied from the probe and form stress magnitude information; a processor for forming elastic images based on the received signals and the stress magnitude information; and a display unit for displaying the elastic images.

Any reference in this specification to “one embodiment,” “an embodiment,” “example embodiment,” etc. means that a particular feature, structure or characteristic described in connection with the embodiment is included in at least one embodiment of the present invention. The appearances of such phrases in various places in the specification are not necessarily all referring to the same embodiment. Further, when a particular feature, structure or characteristic is described in connection with any embodiment, it is submitted that it is within the purview of one skilled in the art to effect such feature, structure or characteristic in connection with other ones of the embodiments.

Although the embodiments have been described with reference to a number of illustrative embodiments thereof, it should be understood that numerous other modifications and embodiments can be devised by those skilled in the art that will fall within the spirit and scope of the principles of this disclosure. More particularly, numerous variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

As an example, although the embodiments describe that the stress sensing unit 120, the supporting unit 130 and the moving unit 140 are separated from the ultrasound diagnosis unit 150, the present invention is not limited to such embodiments. For example, the stress sensing unit 120, the supporting unit 130, the moving unit 140 and the ultrasound diagnosis unit 150 may be formed as a single body in other embodiments.

What is claimed is:

1. An ultrasound system, comprising:
a probe for transmitting ultrasound signals to a target and receiving the ultrasound signals reflected from the target, the probe being further configured to apply stress to the target;
a stress sensing unit disposed at an opposite side of the probe to support the target and sense a magnitude of the stress applied from the probe, the stress sensing unit being further configured to form stress magnitude information;
a processor for forming elastic images based on the received signals and the stress magnitude information; and
a display unit for displaying the elastic images.

2. The ultrasound system of claim 1, further comprising:
a supporting unit for supporting the stress sensing unit; and
a moving unit for moving the supporting unit.
